(19) United States
(12) Patent Application Publication
(10) Pub. No.: US 2016/0250166 A1
(43) Pub. Date: Sep. 1, 2016

(54) METHOD FOR TREATING CHIKUNGUNYA VIRUS INFECTION

(71) Applicant: Chung Yuan Christian University, Jhongli City (TW)

(72) Inventors: Szu-Cheng KUO, New Taipei City (TW); Tzong-Yuan WU, Panchiao City (TW); Chang-Chi LIN, New Taipei City (TW); Yu-Ming WANG, New Taipei City (TW); Ying-Ju CHEN, Shinyuan Township (TW)

(21) Appl. No.: 15/150,852

(22) Filed: May 10, 2016

Related U.S. Application Data
(63) Continuation-in-part of application No. 14/274,239, filed on May 28, 2014, now Pat. No. 9,381,175.

(51) Int. Cl. A61K 31/185; A61K 9/00

(52) U.S. Cl. CPC A61K 31/185; A61K 9/0019; A61K 9/0053

(57) ABSTRACT

Disclosed herein is a method for the treatment of an infection with, or disease caused by, Chikungunya virus in a subject. The method includes administering to the subject a therapeutically effective amount of suramin as the active agent.

### CROSS-REFERENCE TO RELATED APPLICATIONS

This application is a Continuation-in-Part of the co-pending U.S. patent application Ser. No. 14/274,239, filed on May 9, 2014; the entirety of which is incorporated herein by reference for all purposes.

### BACKGROUND OF THE INVENTION

1. Field of the Invention

The present disclosure relates to the treatment of a virus infection. More particularly, the disclosed invention relates to the treatment of Chikungunya virus infection.

2. Description of Related Art

Chikungunya is a mosquito-borne virus mainly transmitted to vertebrates by Aedes mosquitoes such as Aedes albopictus and Aedes aegypti mosquitoes. Chikungunya virus (CHIKV, family Togaviridae, genus Alphavirus) has a positive sense single stranded RNA genome. Alphaviruses use receptor-mediated endocytic uptake and low pH-triggered membrane fusion to deliver their RNA genomes into the cytoplasm where productive replication occurs.

Chikungunya infection (also known as Chikungunya fever) was first identified in Tanzania and Uganda in 1953. Since then, re-emergences of chikungunya infection outbreak have taken place in Africa, Southeast Asia, the Indian subcontinent and the Indian Ocean. Since the 2005 CHIKV outbreak that occurred on Reunion Island, CHIKV disease incidence and persistent CHIKV-induced RA-like symptom. In August 2007, the first outbreak in European continent was documented in Italy with 217 laboratory-confirmed cases. This outbreak was the first one reported in a temperate climate country. Currently, CHIKV has been identified in more than 45 countries. From December 2013 to January 2016, more than 1.5 million confirmed or suspect cases of CHIKV have been reported in America Thus, Chikungunya have been a burden on public health

The symptoms of Chikungunya infection include sudden onset of fever, joint pain, muscle pain, headaches, nausea, vomiting, and nose and gum bleeding. Possible, but relatively rare complications include gastro-intestinal complications, cardiovascular decompensation, and meningo-ecephalitis. Averagely, the symptoms appear on 4 to 7 days after being bitten by an infected mosquito. While most patients usually recover after days to weeks, some may develop chronic arthritis. Death related to Chikungunya infection has been reported mainly in aged patients or patients with weakened immune systems.

Due to the lack of specific anti-CHIKV drugs, the nosological approach is presently the only treatment option for post-CHIKV rheumatic disorders. Currently, alphaviral arthritides can be relieved with analgesics and/or nonsteroidal anti-inflammatory drugs (NSAIDs). Treating CHIKV-induced RA-like arthritis with NSAIDs alone or conjugation with steroid leads to a positive clinical response. CHIKV-induced RA-like arthritis can also be efficiently treated with methotrexate of disease-modifying anti-rheumatic drug (DMARD) therapy. However, the immunosuppressive activities of the aforementioned drugs should be considered during the acute phase of CHIKV infection.

In view of the foregoing, there exists a need in the art for providing a measure for treating and/or preventing Chikungunya infection.

### SUMMARY

The following presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later.

In one aspect, the present disclosure is directed to a method for the treatment of an infection with, or a disease caused by, CHIKV in a subject. In particular, the method is useful in inhibiting the infection or replication of CHIKV in a subject in need of such treatment.

According to one embodiment of the present disclosure, the method comprises administering to the subject a therapeutically effective amount of suramin and a pharmaceutically acceptable excipient to inhibit the infection or replication of CHIKV in the subject.

In optional embodiments, the suramin is administered before and/or after the Chikungunya virus infection.

In various embodiments of the present disclosure, the subject can be a human subject. According to various embodiments of the present disclosure, the suramin is injected intraperitoneally, intravenously or intramuscularly in a solution. Alternatively, the suramin is administered orally in a liquid, solid, or semi-solid dosage form.

Many of the attendant features and advantages of the present disclosure will becomes better understood with reference to the following detailed description considered in connection with the accompanying drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The present description will be better understood from the following detailed description read in light of the accompanying drawings, where:

FIG. 1A is a diagram summarizing the anti-CHIKV activity of suramin in mice, according to Example 1 of the present disclosure;

FIG. 1B is a diagram illustrating foot swelling over time in mice after CHIKV inoculation, according to Example 1 of the present disclosure;

FIG. 1C is a diagram summarizing the effect of suramin treatment on CHIKV-induced foot swelling, according to Example 1 of the present disclosure;

FIG. 2A provides microscopic photographs showing the effect of suramin treatment on CHIKV-induced inflammation, according to Example 1 of the present disclosure;

FIG. 2B is a histogram summarizing the histological evaluation of the effect of suramin treatment on CHIKV-induced inflammation, according to Example 1 of the present disclosure;

FIG. 3A provides microscopic photographs showing the specificity of immunohistochemical analysis of E2 viral antigen expression in CHIKV-infected mice, according to Example 1 of the present disclosure;

FIG. 3B provides microscopic photographs showing the effect of suramin treatment on viral antigen expression in CHIKV-infected mice, according to Example 1 of the present disclosure;

FIG. 3C is a histogram summarizing the intensity of CHIKV E2 signal in CHIKV-infected, according to Example 1 of the present disclosure;

FIG. 4 provides microscopic photographs showing the effect of suramin treatment on CHIKV-induced cartilage damage at 7 dip, according to Example 1 of the present disclosure;

FIG. 5 is a line graph summarizing the average group weight of CHIKV-infected mice with or without suramin treatment, according to Example 1 of the present disclosure;

FIG. 6A is a diagram summarizing the dosage effect and time meanings of suramin treatment on CHIKV-induced foot swelling, according to Example 2 of the present disclosure;

FIG. 6B is a diagram summarizing the dosage and time meanings effects of suramin treatment on viral burdens, according to Example 2 of the present disclosure; and

FIG. 7 is a line graph summarizing the average group weight of CHIKV-infected mice in dosage and time meanings assays, according to Example 2 of the present disclosure.

### DESCRIPTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

For convenience, certain terms employed in the specification, examples and appended claims are collected here. Unless otherwise defined herein, scientific and technical terminologies employed in the present disclosure shall have the meanings that are commonly understood and used by one of ordinary skill in the art.

Unless otherwise required by context, it will be understood that singular terms shall include plural forms of the same and plural terms shall include the singular. Specifically, as used herein and in the claims, the singular forms “a” and “an” include the plural reference unless the context clearly indicated otherwise. Also, as used herein and in the claims, the terms “at least one” and “one or more” have the same meaning and include one, two, three, or more. Furthermore, the phrases “at least one of A, B, and C”, “at least one of A, B, or C” and “at least one of A, B and/or C,” as use throughout this specification and the appended claims, are intended to cover A alone, B alone, C alone, A and B together, B and C together, A and C together, as well as A, B, and C together.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art. Other than in the operating/working examples, or unless otherwise expressly specified, all of the numerical ranges, amounts, values and percentages such as those for quantities of materials, durations of times, temperatures, operating conditions, ratios of amounts, and the likes thereof disclosed herein should be understood as modified in all instances by the term “about.” Accordingly, unless indicated to the contrary, the numerical parameters set forth in the present disclosure and attached claims are approximations that can vary as desired. At the very least, each numerical parameter should at least be construed in light of the number of reported significant digits and by applying ordinary rounding techniques. Ranges can be expressed herein as from one endpoint to another endpoint or between two endpoints. All ranges disclosed herein are inclusive of the endpoints, unless specified otherwise.

The terms “treatment” and “treating” are used herein broadly to include preventative (e.g., prophylactic), curative, or palliative treatment that results in a desired pharmaceutical and/or physiological effect. Preferably, the effect is therapeutic in terms of partially or completely curing or preventing Chikungunya infection. Also, the terms “treatment” and “treating” as used herein refer to application or administration of suramin or a pharmaceutical composition comprising the same to a subject, who has Chikungunya infection, a symptom of Chikungunya infection, a disease or disorder secondary to Chikungunya infection, or a predisposition toward Chikungunya infection, with the purpose to partially or completely alleviate, ameliorate, relieve, delay the onset of, inhibit the progression of, reduce the severity of, and/or reduce the incidence of one or more symptoms or features of Chikungunya infection. Generally, a “treatment” includes not just the improvement of symptoms or decrease of markers of the disease, but also a cessation or slowing of progress or worsening of a symptom that would be expected in absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, and remission (whether partial or total), whether detectable or undetectable.

Through the present application, the terms “application” and “administration” are used interchangeably to mean the application of suramin or a pharmaceutical composition comprising the same to a subject in need of the treatment.

The term “therapeutically effective amount” as used herein refers to the quantity of a component (such as suramin) which is sufficient to yield a desired therapeutic response. A therapeutically effective amount is also one in which any toxic or detrimental effects of the compound or composition are outweighed by the therapeutically beneficial effects. The specific effective or sufficient amount will vary with such factors as the particular condition being treated, the physical condition of the patient (e.g., the patient's body mass, age, or gender), the type of mammal or animal being treated, the duration of the treatment, the nature of concurrent therapy (if any), and the specific formulations employed and the structure of the compounds or its derivatives. Effective amount may be expressed, for example, in grams, milligrams or micrograms or as milligrams per kilogram of body weight (mg/kg).

The term “excipient” as used herein means any inert substance (such as a powder or liquid) that forms a vehicle/carrier for suramin. The excipient is generally safe, non-toxic, and in a broad sense, and may also include any known substance in the pharmaceutical industry useful for preparing pharmaceutical compositions such as, fillers, diluents, agglutinants, binders, lubricating agents, glidants, stabilizer, colorants, wetting agents, disintegrants, and etc.

As used herein, a “pharmaceutically acceptable excipient” is one that is suitable for use with the subjects without undue adverse side effects (such as toxicity, irritation, and allergic response) commensurate with a reasonable benefit/risk ratio. Also, each excipient must be “acceptable” in the sense of being compatible with the other ingredients of the pharmaceutical composition. The excipient can be in the form of a solid, semi-solid, or liquid diluent, cream or a capsule.

The term “subject” refers to a mammal including the human species that is treatable with suramin. The term “subject” is intended to refer to both the male and female gender unless one gender is specifically indicated. Examples of a “subject” or “patient” include, but are not limited to, a human, rat, mouse, guinea pig, monkey, pig, goat, cow, horse, dog, cat, bird and fowl. In an exemplary embodiment, the patient is a human. The term “mammal” refers to all members of the class Mammalia, including humans, primates, domestic and farm animals, such as rabbit, pig, sheep, and cattle; as well as zoo, sports or pet animals; and rodents, such as mouse and rat. The term “non-human mammal” refers to all members of the class Mammalis except human.

The present invention is based, at least, on the finding that the suramin could be used as the sole active agent for the treatment of Chikungunya infection. Using a bi-cistronic baculovirus expression system capable of co-expressing EGFP and CHIKV structural protein in Sf21 cells, various compounds were screened for their ability un specifically inhibiting the membrane fusion of the CHIKV. The screening result, as provided below, indicates that several candidate compounds (including heparin and dextran sulfate) can inhibit the membrane fusion of other alphaviruses such as Venezuelan equine encephalitis virus, yet, they are not effective to inhibit the membrane fusion of CHIKV. However, suramin was found to block the membrane fusion of the CHIKV. Accordingly, suramin was subjected to in vitro assay. The result thereof establishes that suramin inhibits the infection and/or proliferation of the CHIKV.

In one aspect, the present disclosure is directed to a method for the treatment of an infection with, or a disease caused by, Chikungunya virus in a subject.

According to one embodiment of the present disclosure, the method comprises administering to the subject a therapeutically effective amount of suramin and a pharmaceutically acceptable excipient to inhibit the infection or replication of Chikungunya virus in the subject.

In certain embodiments, the subject is a mouse. In other embodiments, the subject is a human.

According to some embodiments of the present disclosure, the infection or replication of Chikungunya virus is inhibited by suppressing or blocking the membrane fusion and viral releasing of the CHIKV infection. For example, the membrane fusion can be envelope protein-mediated membrane fusion.

In some optional embodiments, the suramin is administered before the Chikungunya infection. In other embodiments, the suramin is administered after the Chikungunya infection. In some other embodiments, the suramin is first administered before the Chikungunya infection, and then administered after the Chikungunya infection. Specifically, according to certain working examples provided below, the suramin is administered once before the Chikungunya infection, and twice after the Chikungunya infection.

In the case where the subject is a mouse (weight range: 0.018-0.033 kg), the therapeutically effective amount of suramin is about 0.1 mg to 5 mg per dose, and preferably, about 0.25 mg to 2 mg per dose. Specifically, the therapeutically effective amount is about 0.1, 0.2, 0.3, 0.4, 0.5, 0.6, 0.7, 0.8, 0.9, 1, 1.5, 2, 2.5, 3, 3.5, 4, 4.5, or 5 mg per dose. The effective amount can also be expressed in mg/kg dose, and accordingly, the therapeutically effective amount of suramin in mice is about 5 mg/kg to 250 mg/kg per dose, and preferably, about 10 mg/kg to 100 mg/kg per dose. Specifically, the therapeutically effective amount is about 5, 10, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250 mg/kg per dose. In particular, the amount used in the working example below on mice (weighing about 18-25 grams) is about 0.25, 0.5, 1, or 2 mg per dose for intraperitoneal injection.

Persons having ordinary skills could calculate the human equivalent dose (HED) for the suramin or a pharmaceutical composition comprising the same based on the animal doses provided herein. For example, one may follow the guidance for industry published by US Food and Drug Administration (FDA) entitled “Estimating the Maximum Safe Starting Dose in Initial Clinical Trials for Therapeutics in Adult Healthy Volunteers” in estimating a maximum safe dosage for use in human subjects. Also, although in the mouse model, suramin is administered by using intraperitoneal delivery, it is feasible for persons having ordinary skill in the art to determine the dosage suitable for use in other delivery routes (such as intramuscular or intravenous injection, or oral administration) in a human subject.

Generally, to convert the mg/kg dose used in a mouse (weight range: 0.018-0.033 kg) to an equivalent dose in an adult human (weight range: 50-80 kg), a standard conversion factor is 0.081. That is, the therapeutically effective amount of suramin for an adult human is approximately 0.4 mg/kg to 20 mg/kg per dose; preferably, about 0.8 mg/kg to 8 mg/kg per dose. Specifically, the therapeutically effective amount of suramin for an adult human is about 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, or 20 mg/kg per dose. For a human weighing about 60 kg, the therapeutically effective amount is about 25 mg to 1000 mg per dose; preferably, about 50 mg to 500 mg per dose. Specifically, the therapeutically effective amount of suramin for an adult human is about 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, or 1000 mg per dose.

According to optional embodiments of the present invention, the suramin can be administered intravenously, intramuscularly, or orally. In some embodiments, the suramin or a pharmaceutical composition comprising the same can be given before the infection of Chikungunya virus as a preventive measure. In other embodiments, the suramin can be given after the infection of Chikungunya virus to inhibit the infection or replication of CHIKV. Still optionally, the suramin is given both before and after the Chikungunya virus, so as to inhibit the infection or replication of CHIKV. According to various embodiments of the present disclosure, the suramin can be administered in a single dose or multiple doses. In the case where the suramin is given in multiple doses, the time interval between two doses is about 1, 2, 3, 4, 5, 6, 7, 8, 9, 10, 11, 12, 13, 14, 15, 16, 17, 18, 19, 20, 21, 22, 23, or 24 hours, or 2, 3, 4, 5, 6, 7 days. Also, when the suramin is given in more than three doses, the time interval between the first two doses and the time interval between the second and third doses can be different, and so on.

According to various embodiments of the present disclosure, the pharmaceutical composition comprising suramin is prepared in accordance with acceptable pharmaceutical procedures, such as those described in Remington: The Science and Practice of Pharmacy, 20^{th} edition, ed. Alfonoso R. Gennaro, Lippincott Williams & Wilkins (2000).

The suramin or pharmaceutical compositions comprising the same may be administered by any suitable route, for example, by oral or parenteral (such as, intravenous, subcutaneous, intramuscular, or intraperitoneal peritumoral injection) administration.

A pharmaceutical composition suitable for oral administration may be any orally acceptable dosage form including capsules, tablets, emulsions and aqueous suspensions, dispersions, and solutions. For example, the present pharmaceutical composition or component(s) thereof may be formulated into tablets containing various excipients such as microcrystalline cellulose, sodium citrate, calcium carbonate, dicalcium phosphate, and glycine; along with various disintegrants such as starch, alginic acid and certain silicates; together with granulation binders like polyvinylpyrrolidone, sucrose, gelatin and acacia. Tablets can additionally be prepared with enteric coatings. Additionally, lubricating agents such as magnesium stearate, sodium lauryl sulfate, and talc may be added in the tablet form. For oral administration in a capsule form, solid fillers (such as, dried corn starch, milk sugar, and high molecular weight polyethylene glycols) may be employed in gelatin capsules. When aqueous or semi-solid dosage forms are desired for oral administration, the present pharmaceutical composition or component(s) thereof may be suspended or dissolved in a suitable solvent, optionally combined with emulsifying or suspending agents.

Regarding parenteral administration, the pharmaceutical compositions may be formulated with a pharmaceutically acceptable excipient such as a sterile aqueous solution, which is preferably isotonic with the body fluid of the recipient. Such formulations may be prepared by dissolving or suspending the active ingredient (i.e., suramin) in water containing physiologically compatible substances such as sodium chloride, glycine and the like, and having a buffered pH compatible with physiological conditions to produce an aqueous solution, and rendering said solution sterile. Other diluents or solvent suitable for manufacturing sterile injectable solution or suspension include, but are not limited to, 1,3-butanediol, mannitol, water, and Ringer's solution. Fatty acids, such as oleic acid and its glyceride derivatives are also useful for preparing injectables, as are natural pharmaceutically-acceptable oils, such as olive oil or castor oil. These oil solutions or suspensions may also contain alcohol diluent or carboxymethyl cellulose or similar dispersing agents. Other commonly used surfactants such as Tweens or Spans or other similar emulsifying agents or bioavailability enhancers that are commonly used in manufacturing pharmaceutically acceptable dosage forms can also be used for the purpose of formulation.

Still optionally, pharmaceutical compositions of the present invention can also comprise various pharmaceutically-acceptable additives well known to the art. Said additives include, but are not limited to, drying agent, anti-itch agents, anti-foaming agents, buffers, neutralizing agents, pH adjusting agents, coloring agents, discoloring agents, emollients, emulsifying agents, emulsion stabilizers, viscosity builders, humectants, odorants, preservatives, antioxidants, chemical stabilizers, thickening agents, stiffening agents, or suspending agents.

The following Examples are provided to elucidate certain aspects of the present invention and to aid those of skilled in the art in practicing this invention. These Examples are in no way to be considered to limit the scope of the invention in any manner. Without further elaboration, it is believed that one skilled in the art can, based on the description herein, utilize the present invention to its fullest extent. All publications cited herein are hereby incorporated by reference in their entirety.

### EXAMPLES

Materials and Methods

Viruses, Cells and Chemical

BHK-21 cells were cultured in Dulbecco's modified Eagle medium (DMEM) with 5% heat inactivated Fetal bovine serum (FBS) and antibiotics under 5% CO₂ at 37° C. C6/36 cells were cultured in RPMI-1640 medium with 10% heat inactivated FBS and antibiotics under 5% CO₂ at 28° C. CHIKV strains 0611aTw (Singapore/0611aTw/2006/FJ807896), 0810bTw (Malaysia/0810bTw/2008/FJ807899), and 0706aTw (Indonesia/0706aTw/2007/FJ807897) strains were amplified and titers were determined by plaque assay in BHK-21 cells. Suramin was purchased from Sigma-Aldrich (catalog #S2671).

Animals

All animals were handled in strict accordance with good animal practice as defined by the Council of Agriculture, Executive Yuan (Taiwan, R. O. C). Protocol involving animals were approved by the Institutional Animal Care and Use Committee of the Institute of Preventive Medicine, National Defense Medical Center. According to relevant regulations in Taiwan, CHIKV is classified as a bio-agent in Risk Group 3. All studies with viable CHIKV were performed in certified BSL-3 laboratories. Biosafety protocols used by this study were approved by the Institutional Biosafety Committees of the Institute of Prevention Medicine, National Defense Medical Center.

CHIKV Challenge Study

C57BL/6JNarl (B6) mice of specific pathogen-free condition were purchased from the National Laboratory Animal Center (Taipei, Taiwan). All mice were female with an age of 4 weeks. Suramin was diluted in normal saline in a ratio of 2.5, 5, 10, or 20 mg/mL. 0.1 ml per dose of suramin solution was given at 4 hours before the infection, 1 day post-infection (dpi) and/or 3 dpi via intraperitoneal injection. 50 μl of CHIKV (10⁵ pfu) were inoculated subcutaneously (s.c.) in the ventral side of the right hind foot (towards the ankle). Submandibular blood was collected at 2 pi for viremia analysis. The height and width of the metatarsal area of the hind feet were measured by using Kincrome digital verniercalipers at 7 dpi. Mice were euthanized with isoflurane anesthesia overdose followed by cervical dislocation. Hind feet from scarified mice were collected for histopathologic analysis at 7 dpi.

Histopathology and Immunohistochemistry (IHC)

Hind feet were fixed in 10% buffered formalin (Electron Microscopy Sciences), decalcified with 15% EDTA in 0.1% phosphate buffer over 10 days and embedded in paraffin wax. Sections of 3 μm-thick were cut and stained with hematoxylin-eosin or Safranin-O/Fast Green dye. Histological evaluation was conducted according to the following a scoring system: 0=no inflammation, 1=minimal inflammatory infiltration, 2=mild infiltration, 3=moderate infiltration with moderate edema, 4=marked infiltration with marked edema, and 5=severe infiltration with edema (n=5 feet per group). For immunohistochemistry analysis, 3 μm-thick sections were deparaffinized with xylene and rehydrated in serial dilutions of ethanol. Antigens were retrieved by immersing tissue sections into epitope retrieval buffer. Endogenous peroxidase activity was blocked with 3% hydrogen peroxide solution, and slides were incubated with protein blocking buffer for 10 minutes. Subsequently, tissue sections were incubated with rabbit anti-CHIKV E2 sera (1/1000) at room temperature for 30 minutes, washed in TBST at pH 7.0, and then developed using the Polink-2 HRP Plus Rabbit DAB Detection System (Golden Bridge International, Inc.) per the manufacturer's protocol. Finally, HRP activity was developed with 3,3-diaminobenzidine (DAB) solution and counterstained with hematoxylin. All pictures (six pictures from 2 feet per group) were taken under the same lighting and magnification parameters with Olympus BX43. The intensity of CHIKV E2 signals was measured with ImageJ. For this, color deconvolution was used to separate DAB stains (CHIKV E2 signals) from hematoxylin stain (whole tissue area). Specifically, each picture was converted to grayscale and adjusted using the Thresholding method, and the areas of DAB and hematoxylin stains were measured (Analyze/Measure). CHIKV E2 expression score was calculated by dividing the total tissue area by the positively stained area.

Measurements of Viremia

Sera from submandibular blood of CHIKV-infected or mock infected with PBS containing 10% culture medium were stored at −70° C. In a 96-well plate, 3×10⁵ C6/36 cells per well were inoculated with 100 μl medium containing a serial 10-fold diluted blood sample in duplicate. At 3 dpi, fixed cells were stained with rabbit anti-CHIKV E2 serum (1/100) and Alex 488 goat ant-rabbit IgG (1/500) (Invitrogen, Molecular Probes, Carlsbad, Calif.). Viral loads were expressed as CCID50/ml of serum.

Statistical Analysis

All statistical analyses were performed using GraphPad Prism version 6.0.1 software. Differences between the mock-treated group and the suramin-treated group were assessed using the student's t test. P values of <0.05 were considered to be statistically significant.

### Example 1

### Suramin Treatment Decreases Viral Burden and Musculoskeletal Lesions in CHIKV-Infected Mice

To assess the anti-CHIKV activity of suramin in vivo, mice (5 per group) were infected with one of the following CHIKV strains: 0611aTw, 0810bTw or 0706aTw. CHIKV-infected mice were either treated with 100 μl normal saline (0810bTw, 0611aTw and 0706aTw) or 2 mg suramin (0810bTw+ suramin, 0611aTw+ suramin and 0706aTw+ suramin) 4-hrs pre-infection; or 24- or 48-hrs post-infection by the ip route. Control mice were injected with 50 μl diluted medium (1/10) into right hind feet, and treated with 100 μl normal saline at the same time course (i.e., 4-hrs pre-infection; or 24- or 48-hrs post-infection by the ip route).

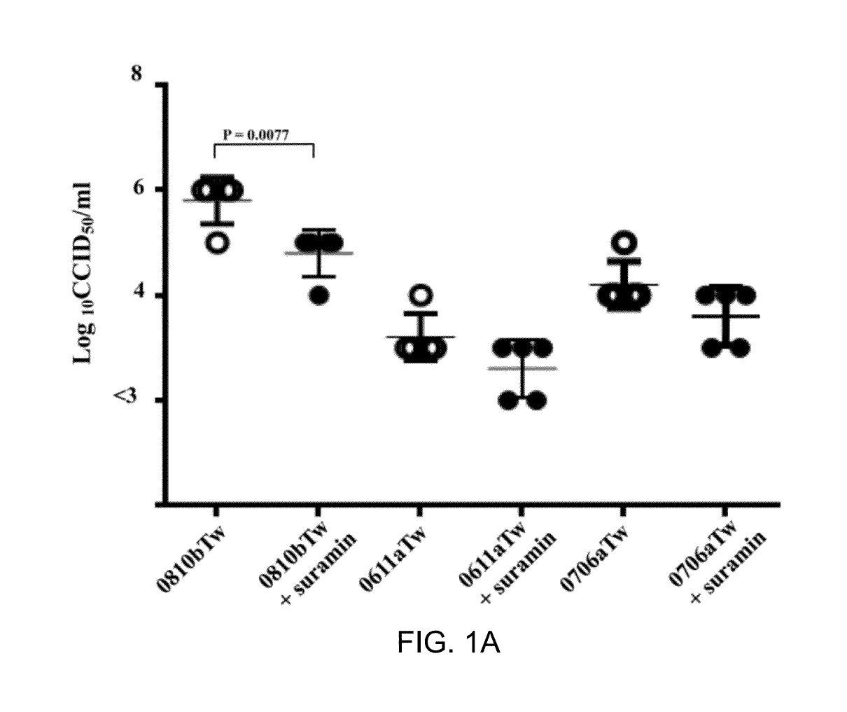

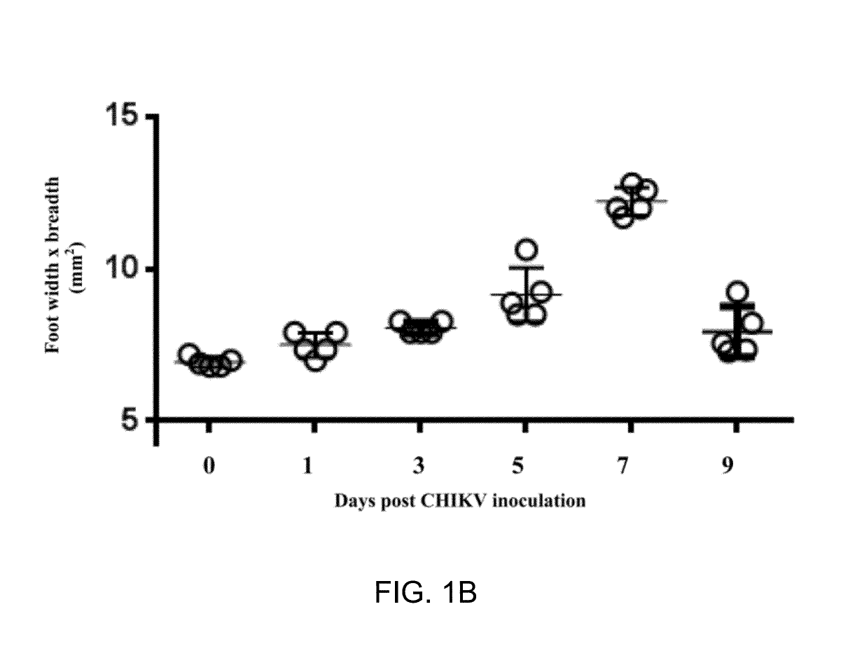

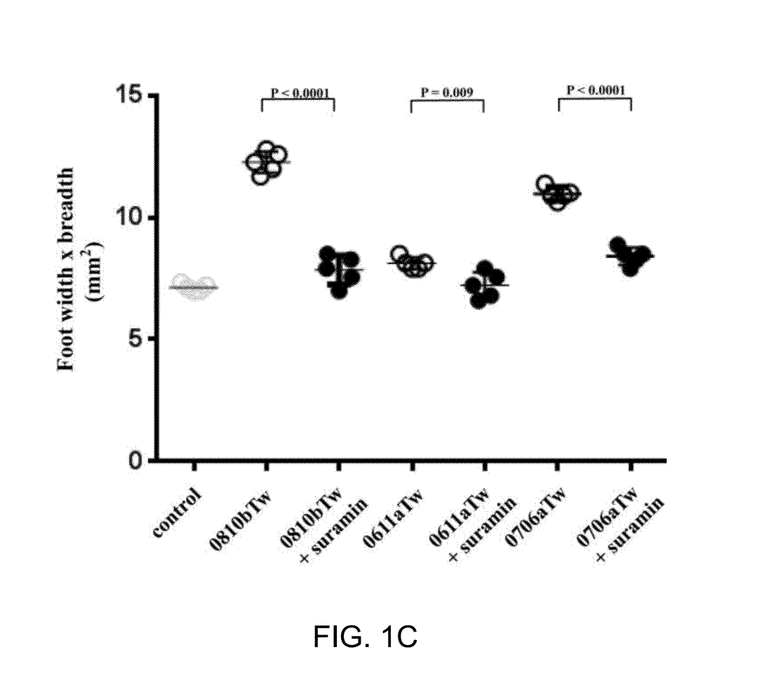

Viremia results, as summarized in FIG. 1A, indicated that viral loads in sera of mock-treated mice infected with the 0810bTw, 0611aTw and 0706aTw CHIKV strains at peak of 2 dpi were 5.8, 3.0, and 4.2 Log₁₀ CCID₅₀/ml, respectively. Viral titers of suramin-treated sera were 5.0, 2.5, and 3.8 Log₁₀ CCID₅₀/ml for 0810bTw, 0611aTw and 0706aTw strains, respectively. Suramin treatment not only resulted in statistically significantly decreases in viral loads for 0810bTw-infected mice, but also substantially decreased viral loads in 0611aTw or 0706aTw-infected mice.

In order to determine the severity of foot swelling induced by CHIKV infection, foot swelling over time in 0810bTw-infected mice was quantified. The results summarized in FIG. 1B indicated that maximum foot swelling occurred at 7 dpi.

To further characterize the therapeutic effects of suramin treatment on CHIKV infections, foot swelling and histopathologic lesions at peak disease (7 dpi) of suramin-treated and mock-treated mice were examined. As shown in FIG. 1 C, paw volumes of mock-treated mice infected with 0810bTw, 0611aTw and 0706aTw were 13, 8.5 and 11 mm², respectively. Strains 0810bTw (13 mm²) and 0706aTw (11 mm²) caused swelling and edema which were much more severe than those associated with 0611aTw strain (8.5 mm²). Interestingly, paw volumes of suramin-treated mice infected with 0611aTw, 0810bTw or 0706aTw were decreased by 38, 12.5 and 22.7% compared to their mock-treated counterparts. Furthermore, suramin decreased the size of hind feet in CHIKV-infected mice to nearly that of control (7 mm²) at 7 dpi.

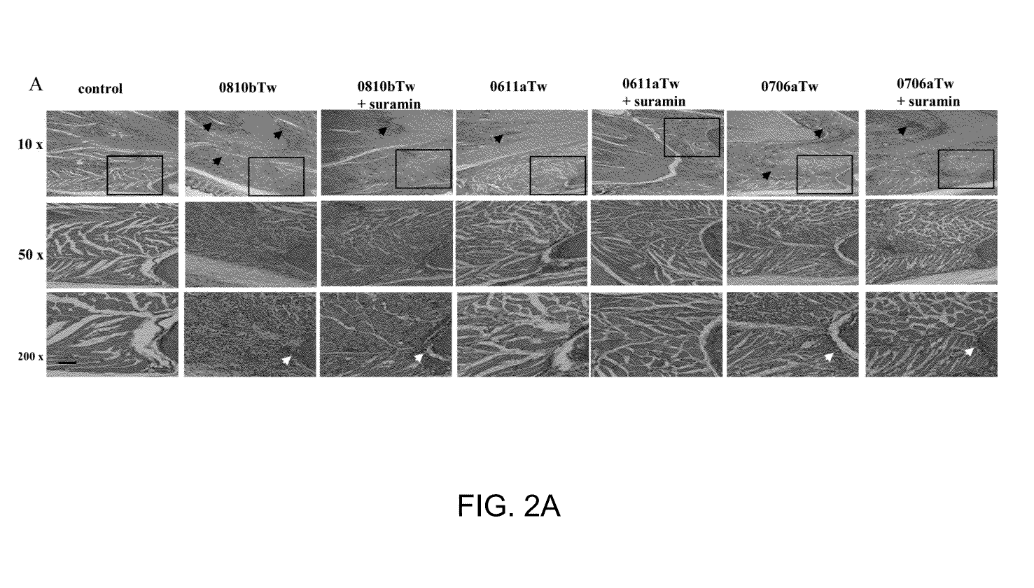

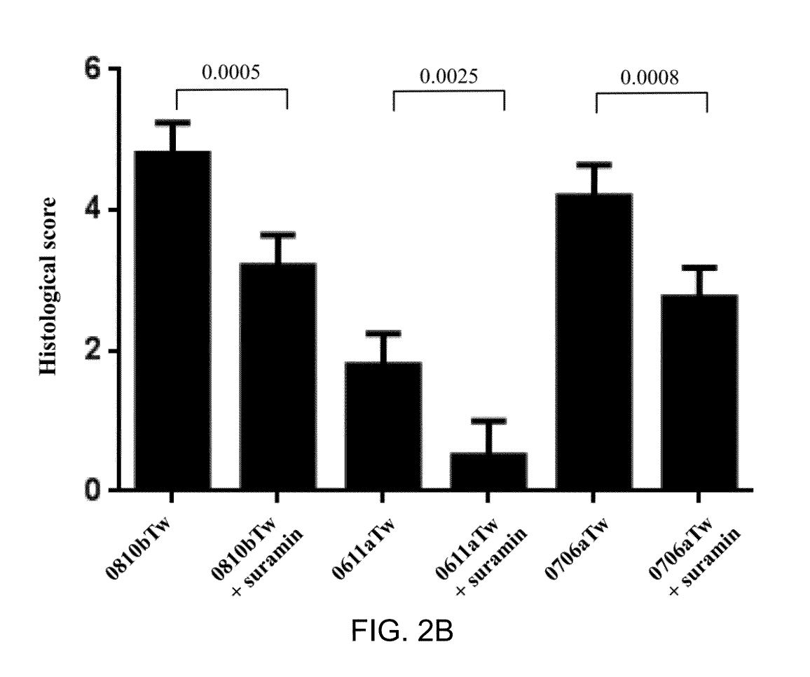

Right hind feet collected at 7 dpi were fixed, decalcified, and stained with hematoxylin-eosin. Microscopic results were provided in FIG. 2A, in which the foci of myositis were indicated by white arrows; tenosynovitis was indicated by black arrows in CHIKV-infected mice, with inflammatory cells present in the tendon capsule; boxed areas in upper panels were shown at higher resolution in bottom panels (×50 and ×200 magnifications; bars, 100 μm). Referring to FIG. 2A, at maximum foot swelling (i.e., 7 dpi), the feet of 0706aTw- and 0810bTw-infected mice had extensive acute lesions including inflammatory infiltrates, subcutaneous edemas, fibrinous exudates, and periostitis. Conversely, infection with the 0611aTw strain only induced mild inflammatory infiltrates. Control mice did not show any inflammatory infiltrates.

In summary, suramin treatment led to a marked reduction in acute foot lesions of mice infected with all three strains of CHIKV compared to mock-treated mice, and histopathological scores revealed that suramin treatment led to a statistically significant reduction in inflammatory infiltrates (FIG. 2B).

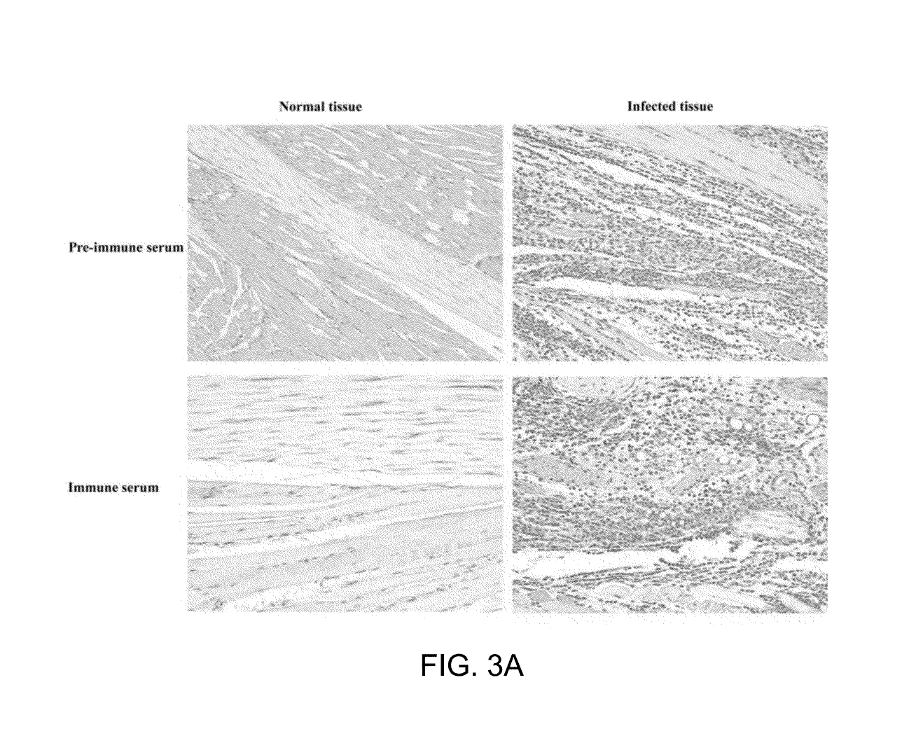

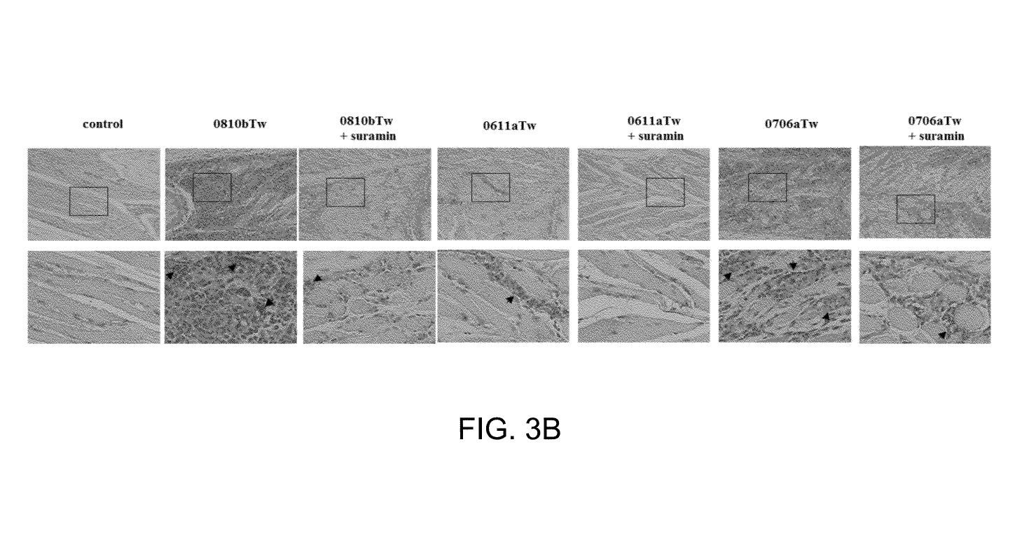

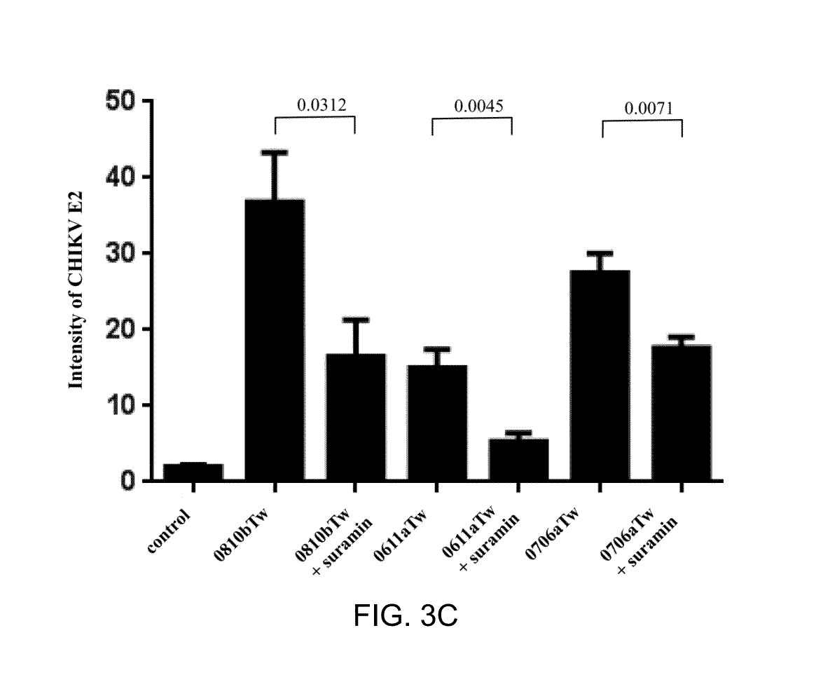

To investigate the effects of suramin treatment on viral infection; IHC was performed to detect the expression of viral antigen (E2 glycoprotein) in infected tissues. The specificity of IHC was demonstrated in FIG. 3A.

Results of immunohistochemical analysis of E2 glycoprotein in feet from suramin-treated or mock-treated mice at 7 dpi were provided in FIG. 3B, in which the foci of E2 glycoprotein were indicated by black arrows; boxed areas in upper panels were shown at higher resolution in bottom panels (×200 magnification). Referring to FIG. 3B, in the feet of CHIKV-infected mice, IHC signals were mainly observed in macrophages, epithelial cells, bone cells, chondrocytes, periosteum and muscle cells. Similar to the histological inflammation scores, a great number of CHIKV E2 glycoprotein was detected in mock-treated mice infected with either 0706aTw or 0810bTw than in 0611aTw-infected mice (FIG. 3B). However, suramin treatment uniformly prohibited the expression of E2 glycoprotein in hind foot tissues of infected mice (FIG. 3C). Therefore, the IHC analysis confirms that suramin treatment significantly inhibits CHIKV replication in vivo.

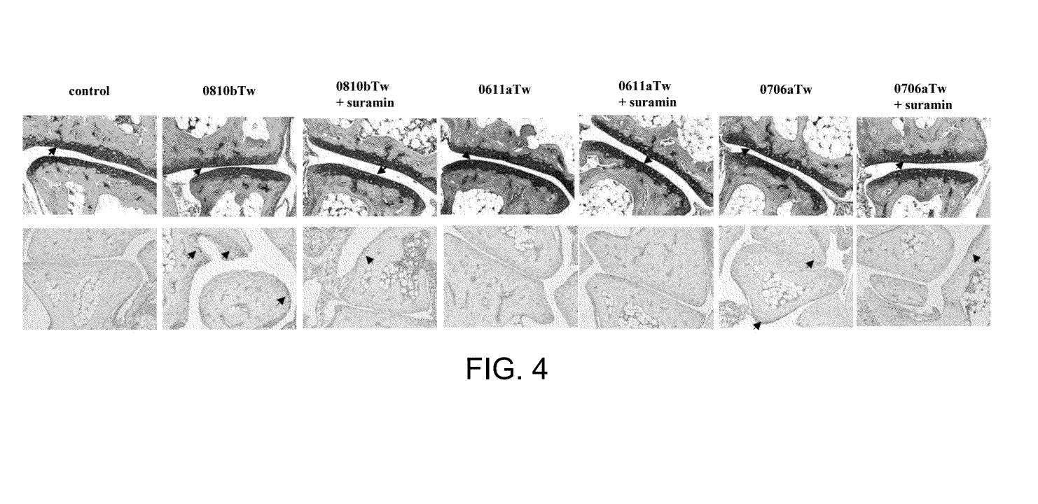

In mice models, RRV and CHIKV infections cause the destruction of cartilage including matrix proteoglycan depletion. Thus, the therapeutic effects of suramin in CHIKV-infected mice were further elucidated by evaluating cartilage integrity. At 7 dpi, IHC and histological evaluations of joint sections stained with Safranin O-fast green, and the results were provided in upper panels of FIG. 4, in which proteoglycan in red were indicated by black arrows. As could be seen in upper panels of FIG. 4, CHIKV strain 0810bTw and 0706aTw led to more severe cartilage destruction and a great number of IHC positive chondrocyte than did the 0611aTw strain. However, suramin treatment markedly restored cartilage integrity and reduced the number of IHC positive chondrocyte in mice infected with 0810bTw and 0706aTw (see, lower panels of FIG. 4, in which the foci of E2 glycoprotein were indicated by black arrows).

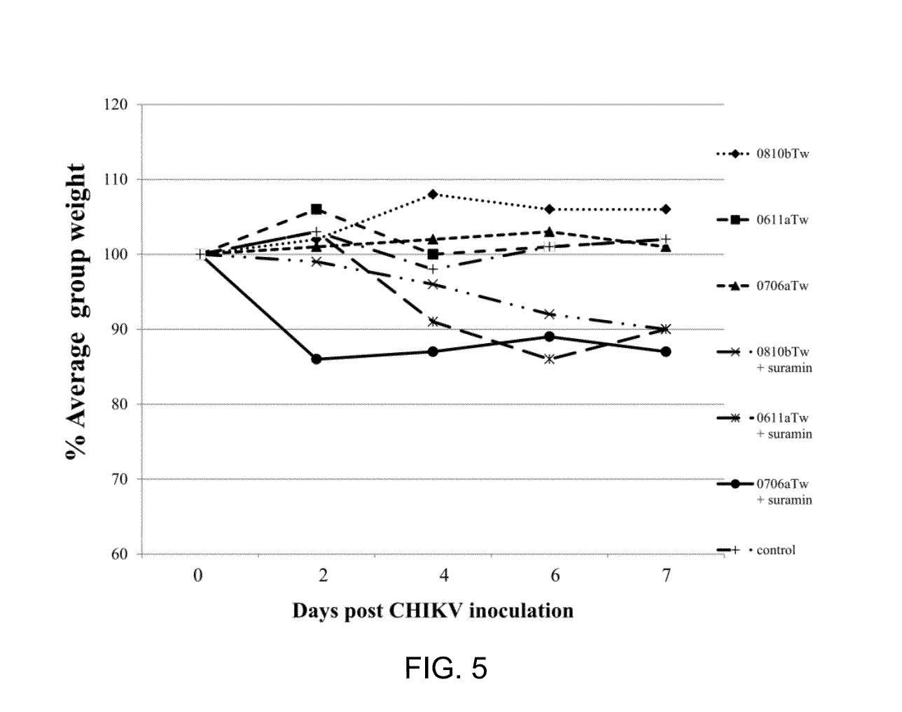

Finally, no detectable weight loss was observed in mock-treated mice and control mice (FIG. 5). Suramin treatment resulted in significant weight loss; however, this effect was temporary.

Taken together, the results described in this section clearly demonstrate that suramin treatment decreases the infectivity of CHIKV and ameliorates CHIKV-induced rheumatic disease in C57BL/6 mice.

### Example 2

### Dose- and Time-Related Evaluations of Suramin Treatment

According to results obtained in Example 1, the most significant therapeutic effects of suramin treatment were observed in 0810bTw-infected mice. Therefore, the CHIKV0810bTw strain was used in this example for dose- and time-related assays. For the dose-related assay, infected mice were treated with three-doses of two-fold serial diluted suramin (2, 1, 0.5, or 0.25 mg) at 4 hrs pre-infection, 1 dpi and 3 dpi, respectively. For the time-related assay, infected mice were treated with a single dose of 2 mg suramin at 4 hrs pre-infection (pre 2 mg), or post-treated with two doses of 2 mg suramin respectively at 1 dpi and 3 dpi (post 2 mg).

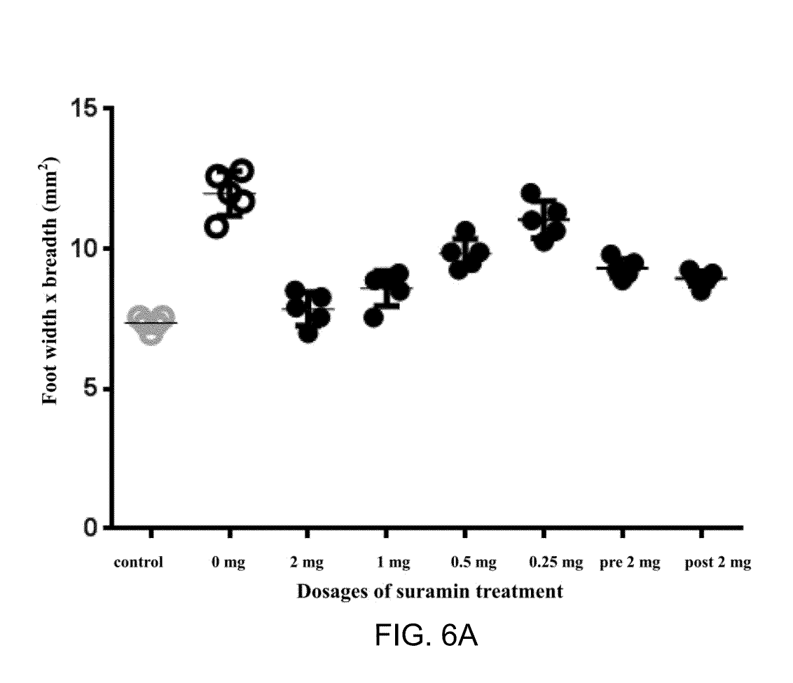

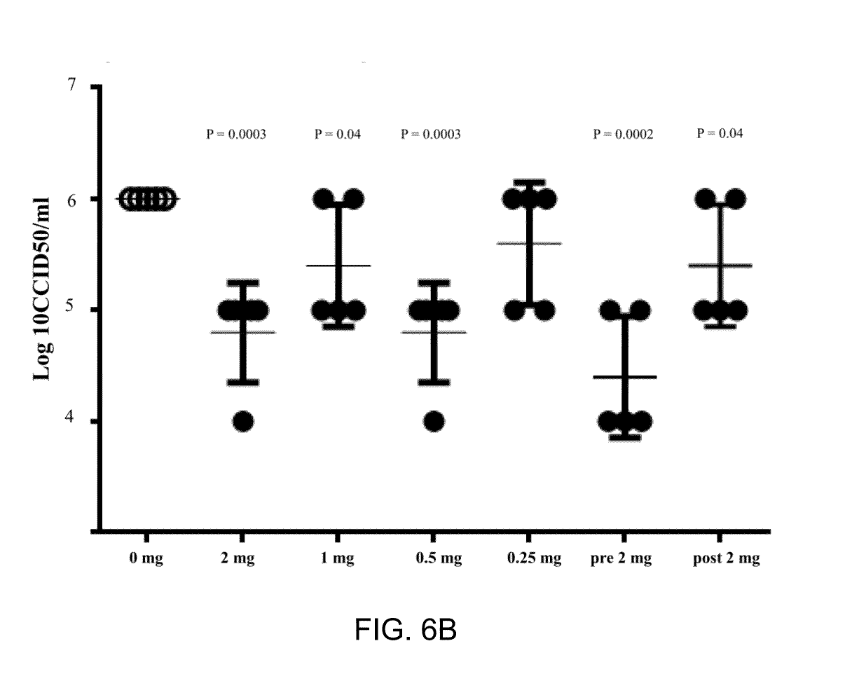

We observe that suramin substantially decreased viremia in dose- and time-related assays (FIG. 6A). Foot swelling under treatment with 2, 1, 0.5, 0.25, pre 2, and post 2 mg suramin were reduced by 34, 29, 18, 8, 22, and 25%, respectively, compared to mock-treated mice (FIG. 6B). Interestingly, a single dosage of 2 mg suramin treatment at 4 hrs pre-infection (pre 2 mg) led to a markedly reduced viral burden.

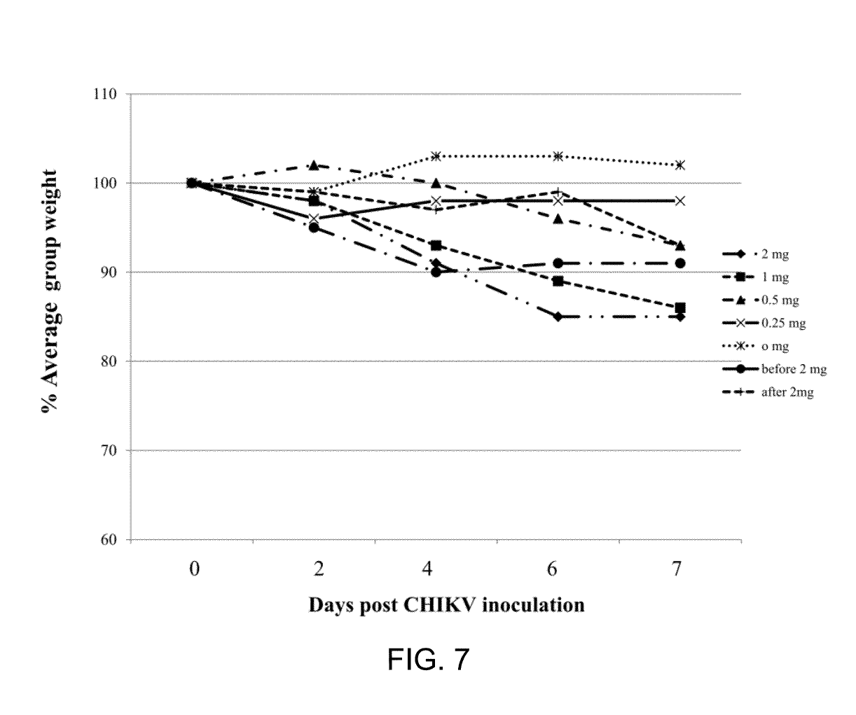

Results from average weight monitoring (FIG. 7) showed dose-dependent weight loss in suramin-treated mice. Notably, while all doses- and time-dependent treatments substantially reduced viral burden and disease score, a single dosage of 2 mg suramin treatment at 4 hrs pre-infection (pre 2 mg) provided the best balance between maximizing therapeutic effects and minimizing weight loss.

Taken together, the experimental data provided herein clearly demonstrate that suramin treatment decreases viral burden and helps mitigate acute disease symptoms in CHIKV-infected mice.

It will be understood that the above description of embodiments is given by way of example only and that various modifications may be made by those with ordinary skill in the art. The above specification, examples, and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

What is claimed is:

1. A method for inhibiting the infection or replication of Chikungunya virus in a subject in need thereof, comprising administering to the subject a therapeutically effective amount of suramin and a pharmaceutically acceptable excipient to inhibit the infection or replication of Chikungunya virus in the subject.

2. The method of claim 1, wherein the suramin is administered before the infection of Chikungunya virus.

3. The method of claim 1, wherein the suramin is administered after the infection of Chikungunya virus.

4. The method of claim 1, wherein the suramin is administered both before and after the infection of Chikungunya virus.

5. The method of claim 1, wherein the subject is a mouse.

6. The method of claim 5, wherein the therapeutically effective amount is 0.1 mg to 5 mg per dose.

7. The method of claim 6, wherein the therapeutically effective amount is 0.25 mg to 2 mg per dose.

8. The method of claim 5, wherein the therapeutically effective amount is 5 mg/kg to 250 mg/kg per dose.

9. The method of claim 8, wherein the therapeutically effective amount is 10 mg/kg to 100 mg/kg per dose.

10. The method of claim 9, wherein the suramin is administered both before and after the infection of Chikungunya virus.

11. The method of claim 9, wherein the suramin is administered in at least one dose before the infection of Chikungunya virus, and in at least two doses after the infection of Chikungunya virus.

12. The method of claim 9, wherein the suramin is administered intraperitoneally.

13. The method of claim 1, wherein the subject is a human.

14. The method of claim 13, wherein the therapeutically effective amount is 25 mg to 1000 mg per dose.

15. The method of claim 14, wherein the therapeutically effective amount is 50 mg to 500 mg per dose.

16. The method of claim 13, wherein the therapeutically effective amount is 0.4 mg/kg to 20 mg/kg per dose.

17. The method of claim 16, wherein the therapeutically effective amount is 0.8 mg/kg to 8 mg/kg per dose.

18. The method of claim 17, wherein the suramin is administered both before and after the infection of Chikungunya virus.

19. The method of claim 18, wherein the suramin is administered in at least one dose before the infection of Chikungunya virus, and in at least two doses after the infection of Chikungunya virus.

20. The method of claim 18, wherein the suramin is administered intravenously, intramuscularly, or orally.
